(19) United States
(12) Plant Patent Application Publication
(10) Pub. No.: US 2018/0359894 P1
(43) Pub. Date: Dec. 13, 2018

(54) Female asparagus plant 'FCE4'

(50) ;

(71) Applicant: The Regents of the University of California, Oakland, CA (US)

(72) Inventors: Neil K. Stone, Temecula, CA (US); Mikeal L. Roose, Riverside, CA (US)

(21) Appl. No.: 15/731,450

(22) Filed: Jun. 9, 2017

(51) Int. Cl. A01H 6/12

(52) U.S. Cl. PLT/260

(57) ABSTRACT

This invention is a new and distinctive female asparagus plant called ‘FCE4’.

Latin name of the genus and species: Asparagus officinalis.

Variety denomination: The variety denomination is ‘FCE4’.

### BACKGROUND OF THE INVENTION

Asparagus is a dioecious species with individual plants being either male or female. Asparagus cultivars that have been most commonly used for fresh market green asparagus in the major growing regions of California include, Atlas, Grande, Ida Lea, UC157, and Depaoli. These cultivars are all produced from crossing a genetically unique male clone with a genetically unique female clone to produce F1 seed.

### BRIEF SUMMARY OF THE INVENTION

‘FCE4’ is a new and genetically distinct female asparagus clone that is used to produce asparagus cultivars that have commercially desirable traits. The closest cultivar to ‘FCE4’ known to the inventor is an unreleased University of California female asparagus clone ‘F600’ (unpatented). ‘FCE4’ can be distinguished from ‘F600’ by its ability to provide a hybrid asparagus cultivar that has spears of a greater weight when crossed with male asparagus ‘M256’ (U.S. Plant Pat. No. 20,629) compared to the weight of the spears from the hybrid cultivar resulting from crossing ‘F600’ with ‘M256’.

### BRIEF DESCRIPTION OF THE DRAWINGS

FIG. 1 provides an illustration of a typical stalk from an asparagus plant. The Figure illustrates the relative positions on the plant and the source of the data presented below. References are made with the use of brackets and letters, the same letters which are also listed in parentheses under the stalk data.

FIG. 2 shows typical foliage of ‘FCE4’. The photograph depicts a four year old ‘FCE4’ plant in a greenhouse at Riverside, Calif.

### DETAILED DESCRIPTION OF THE INVENTION

Pedigree ‘FCE4’ was selected as a single plant in 1999 from a cross between female asparagus clone F109 (U.S. Plant Pat. No. 4,677) and male asparagus clone EC2 (unpatented) and propagated by tissue culture. ‘FCE4’ was selected based on plant vigor, height to the first branch, and its ability to produce a. large number of high quality spears.

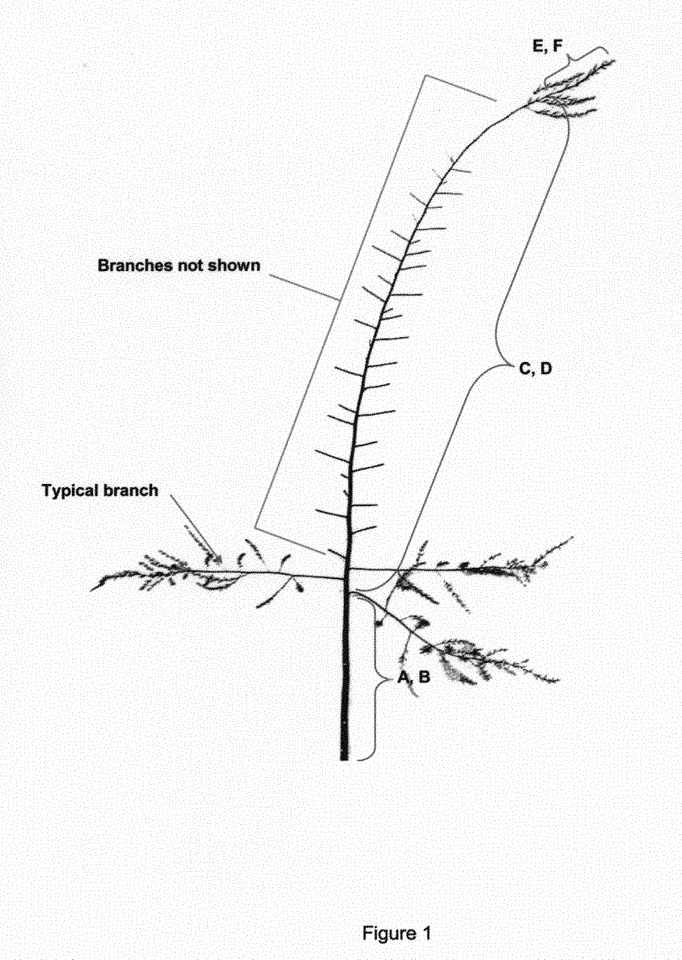

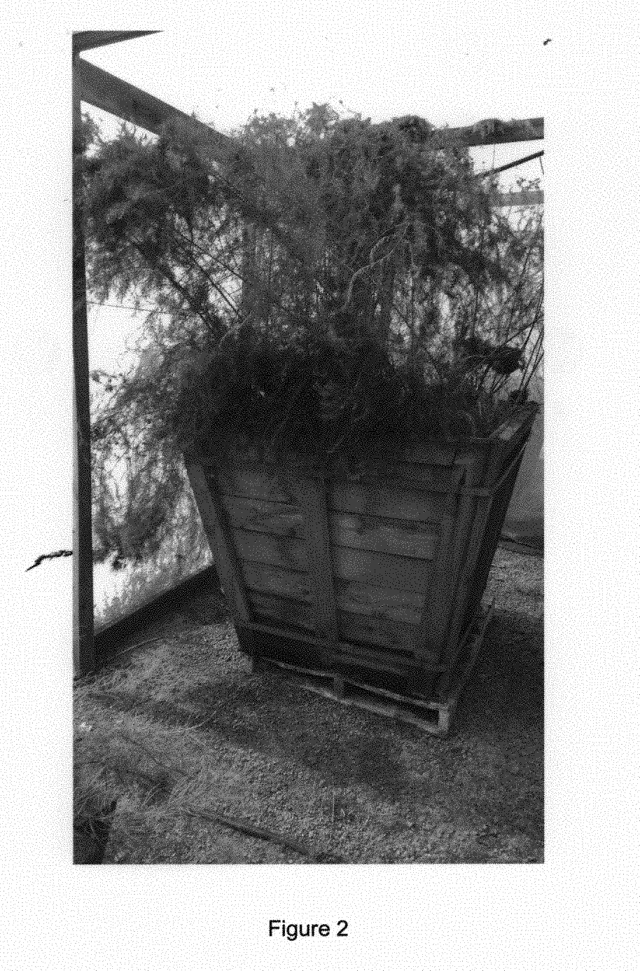

Cultivation ‘FCE4’ can be clonally propagated by crown division. The distinguishing characteristics of ‘FCE4’ are stable and reproduce true to type in successive generations. ‘FCE4’ is usually grown in isolated plantings of clonally propagated plants along with a male clone to produce F1 seeds. Recommended cultural practices for ‘FCE4’ are similar to those of other asparagus plantings for seed production. Plantings can be established using 8-week old seedling transplants or 1-year-old crowns. Performance can be good on soils with low (Riverside, Calif.) or high (Delta regions of California) organic content. Rust and asparagus aphid should be controlled with spray regimes.
Plant characteristics: The attached photograph, FIG. 1, illustrates the relative positions on the plant and the source of the data presented below. References are made with the use of brackets and letters, the same letters which are also listed in parentheses under the stalk data. Typical foliage of FCE4 is shown in FIG. 2. The following measurements were obtained from the longest stalk and are the mean value of measurements on five separate plants unless otherwise indicated. The colors presented below refer to colors in The Royal Horticultural Society color chart and are the most common colors observed among the plants measured.
Stalk description:
   Number of nodes below the first branch (A).—22.2.
   Number of cm from crown to the first branch (B).—37.
   Number of branches (C).—36.2.
   Number of cm between first and last branch (D).—43.9.
   Internode length in cm between branches (D/(C-1)).—1.25.
   Number of cladophyll nodes beyond the last branch (E).—28.0.
   Number of cm beyond the last branch (F).—7.9.
   Internode length in cm beyond the last branch (F/(E-1)).—0.29.
   Largest stalk diameter in mm.—9.86.
   Mean diameter of three largest stalks in mm.—9.83.
   Number of stalks.—7.0.
   Stalk vigor index (stalk number)(mean diameter)2.—674.08.
   Mature stalk color, bloom removed.—144B.
Flower description:
   Petal tip color (four plants).—Yellow 157D.
   Petal base color (four plants).—Yellow-green 146D.
   Flower length in mm.—4.33.
   Flower width at midpoint in mm.—1.98.
Cladophyll description:
   Number per node.—5.4.
   Length in mm.—19.92.
   Width in mm.—0.22.

What is claimed is:

1. A new and distinct female asparagus plant having the characteristics essentially as described and illustrated herein.
